(19) United States
(12) Patent Application Publication
(10) Pub. No.: US 2008/0226551 A1
(43) Pub. Date: Sep. 18, 2008

(54) Transport Molecules Using Reverse Sequence HIV-TAT Polypeptides

(75) Inventors: Jacob M. Waugh, Mountain View, CA (US); Jae Hoon Lee, Union City, CA (US)

Correspondence Address: KING & SPALDING, 1185 AVENUE OF THE AMERICAS, NEW YORK, NY, 10036-4003, US

(73) Assignee: REVANCE THERAPEUTICS, INC., Mountain View, CA (US)

(21) Appl. No.: 11/954,885

(22) Filed: Dec. 12, 2007

Related U.S. Application Data
(60) Provisional application No. 60/882,639, filed on Dec. 29, 2006.

(51) Int. Cl. A61K 51/00; C07K 7/00; C12N 11/02; A61P 43/00; A61K 49/00; C07H 21/00

(52) U.S. Cl. 424/1.69; 530/328; 435/177; 536/23.1; 424/9.3

(57) ABSTRACT

This invention relates to novel transport molecules that comprise a polypeptide comprising amino acid residues arranged in a sequence that is the reverse-sequence of basic portion of the HIV-TAT protein. The novel transport polypeptides are useful for transmembrane or intracellular delivery of cargo molecules, non-limiting examples of which include polypeptides and nucleic acids. The novel transport polypeptides may be covalently or non-covalently bound to the cargo modules.

### CROSS-REFERENCES TO RELATED APPLICATIONS

This application claims priority to U.S. Provisional Application Ser. No. 60/882,639, filed Dec. 29, 2006, the contents of which are incorporated herein by reference in its entirety.

### TECHNICAL FIELD OF THE INVENTION

This invention relates to novel transport molecules that comprise a polypeptide having amino acid residues arranged in a sequence that is the reverse-sequence of the basic portion of the HIV-TAT protein. The novel transport molecules are useful for transmembrane or intracellular delivery of cargo molecules, non-limiting examples of which include polypeptides and nucleic acids. The novel transport molecules may be covalently or non-covalently bound to the cargo molecules. The reduced size of the preferred transport molecule of this invention also minimizes interference with the biological activity of the cargo molecule.

### BACKGROUND OF THE INVENTION

Transmembrane or intracellular delivery of diagnostic or therapeutic agents is often complicated by the inability of such agents to reach the tissues or intracellular sites of interest. This complication may arise, in part, because the membrane organism have evolved to keep out external compounds as a way of protecting the organism.

Consider, for example, the complex structure of human skin, which protects the body's organs from external environmental threats and acts as a thermostat to maintain body temperature. Skin consists of several different layers, each with specialized functions. The major layers include the hypodermis, the dermis and the epidermis. The hypodermis is the deepest layer of the skin. It acts both as an insulator for body heat conservation and as a shock absorber for organ protection (Inlander, Skin, New York, N.Y.: People's Medical Society, 1-7 (1998)). In addition, the hypodermis also stores fat for energy reserves. The pH of skin is normally between 5 and 6. This acidity is due to the presence of amphoteric amino acids, lactic acid, and fatty acids from the secretions of the sebaceous glands. The term “acid mantle” refers to the presence of the water-soluble substances on most regions of the skin. The buffering capacity of the skin is due in part to these secretions stored in the skin's horny layer.

The dermis, which lies above the hypoepidermis, is 1.5 to 4 millimeters thick. It is the thickest of the three layers of the skin. In addition, the dermis is also home to most of the skin's structures, including sweat and oil glands (which secrete substances through openings in the skin called pores, or comedos), hair follicles, nerve endings, and blood and lymph vessels (Inlander, Skin, New York, N.Y.: People's Medical Society, 1-7 (1998)). However, the main components of the dermis are connective tissue such as collagen and elastin.

The epidermis is a stratifying layer of epithelial cells that overlies the dermis and is the topmost layer of skin. The epidermis is only 0.1 to 1.5 millimeters thick (Inlander, Skin, New York, N.Y.: People's Medical Society, 1-7 (1998)), consists of keratinocytes, is divided into several layers based on their state of differentiation. The epidermis can be further classified into the stratum corneum and the viable epidermis, which consists of the granular melphigian and basal cells.

One significant problem in applying physiologically active agents topically or transdermally is that skin is an effective barrier to penetration. The oily nature of the stratum corneum and the tight compaction of its cells provide an effective barrier against gaseous, solid or liquid chemical agents, whether used alone or in water or in oil solutions. Thus, the stratum corneum frustrates efforts to apply therapeutic, cosmetic, or diagnostic agents topically to local areas of the body. This is problematic, because many physiologically active agents ideally should be applied topically in a localized area to achieve sufficiently high local concentrations of the agent to have a therapeutic benefit, without systemic overdose. Additionally, often absorption of a therapeutic or diagnostic agent via gastrointestinal tract is undesirable because it can lead to unwanted chemical alteration of the agent via normal metabolic processes.

Besides macroscopic structures such as skin, cells are generally impermeable or nearly impermeable to many therapeutic of diagnostic agents, particularly if the agents are macromolecules, such as proteins and nucleic acids. Moreover, some small molecules enter living cells at very low rates. The lack of means for delivering macromolecules into cells in vivo has been an obstacle to the therapeutic, prophylactic and diagnostic use of a potentially large number of therapeutic and diagnostic agents having intracellular sites of action, such as proteins and nucleic acids.

Various methods have been developed for delivering macromolecules into cells in vitro. A list of such methods includes electroporation, membrane fusion with liposomes, high velocity bombardment with DNA-coated microprojectiles, incubation with calcium-phosphate-DNA precipitate, DEAE-dextran mediated transfection, infection with modified viral nucleic acids, and direct micro-injection into single cells. These in vitro methods typically deliver the nucleic acid molecules into only a fraction of the total cell population, and they tend to damage large numbers of cells. Experimental delivery of macromolecules into cells in vivo has been accomplished with scrape loading, calcium phosphate precipitates and liposomes. However, these techniques have, to date, shown limited usefulness for in vivo cellular delivery. Moreover, even with cells in vitro, such methods are of extremely limited usefulness for delivery of proteins.

General methods for efficient delivery of biologically active proteins into intact cells, in vitro and in vivo, are needed. (L. A. Sternson, “Obstacles to Polypeptide Delivery”, Ann. N.Y. Acad. Sci, 57, pp. 19-21 (1987)). Chemical addition of a lipopeptide (P. Hoffmann et al., “Stimulation of Human and Murine Adherent Cells by Bacterial Lipoprotein and Synthetic Lipopeptide Analogues”, Immunobiol., 177, pp. 158-70 (1988)) or a basic polymer such as polylysine or polyarginine (W.-C. Chen et al., “Conjugation of Poly-L-Lysine Albumin and Horseradish Peroxidase: A Novel Method of Enhancing the Cellular Uptake of Proteins”, Proc. Natl. Acad. Sci. USA, 75, pp. 1872-76 (1978)) have not proved to be highly reliable or generally useful. Folic acid has been used as a transport moiety (C. P. Leamon and Low, Delivery of Macromolecules into Living Cells: A Method That Exploits Folate Receptor Endocytosis”, Proc. Natl. Acad. Sci. USA, 88, pp. 5572-76 (1991)). Evidence was presented for internalization of folate conjugates, but not for cytoplasmic delivery. Given the high levels of circulating folate in vivo, the usefulness of this system has not been fully demonstrated. Pseudomonas exotoxin has also been used as a transport moiety (T. I. Prior et al., “Barnase Toxin: A New Chimeric Toxin Composed of Pseudomonas Exotoxin A and Barnase”, Cell, 64, pp. 1017-23 (1991)). The efficiency and general applicability of this system for the intracellular delivery of biologically active cargo molecules is not clear from the published work, however.

One previously reported method for intracellular delivery of certain classes of therapeutic agents involves using transport agents that contain basic region of the HIV-TAT protein for intracellular delivery of certain classes of compounds. See, for example, U.S. Pat. Nos. 5,652,122; 5,670,617; 5,674,980; 5,747,641; 5,804,604; and 6,316,003. Additionally, it has been reported that purified human immunodeficiency virus type-1 (“HIV”) TAT protein is taken up from the surrounding medium by human cells growing in culture (A. D. Frankel and C. O. Pabo, “Cellular Uptake of the TAT Protein from Human Immunodeficiency Virus”, Cell, 55, pp. 1189-93 (1988)). Generally, the TAT protein trans-activates certain HIV genes and is essential for viral replication. The full-length HIV-1 TAT protein has 86 amino acid residues. The HIV TAT gene has two exons. TAT amino acids 1-72 are encoded by exon 1, and amino acids 73-86 are encoded by exon 2. The full-length TAT protein is characterized by a basic region which contains two lysines and six arginines (amino acids 49-57) and a cysteine-rich region that contains seven cysteine residues (amino acids 22-37). In particular, the basic region (i.e., amino acids 49-57) is thought to be important for nuclear localization. (Ruben, S. et al., J. Virol. 63: 1-8 (1989); Hauber, J. et al., J. Viral. 63 1181-1187 (1989).

### SUMMARY OF THE INVENTION

Whereas the basic region of HIV-TAT has been previously used to increase intracellular delivery of certain classes of molecules, this invention is based on the unexpected finding that the reverse sequence of the basic region of HIV-TAT can be used to increase transmembrane or intracellular delivery of cargo molecule, as defined herein. As a result of this finding, this invention provides novel transport molecules that are capable of increasing the transmembrane or intracellular penetration of cargo molecules. Thus, the transport molecules of present invention can be used to deliver a cargo molecule across membrane (e.g., transdermally) or through a cell membrane into eukaryotic cells (e.g., into the cell nucleus or cytoplasm), either in vitro or in vivo. This invention further relates to covalently or non-covalently bound conjugates of a transport molecule and a cargo molecule.

Additionally, this invention provides a method of using the novel transport molecules of the invention to increase the transmembrane or intracellular penetration of cargo molecules. This method is particularly suited for cargo molecules that either (1) are not inherently capable of entering target cells, cell nuclei, or membranes, or (2) are not inherently capable of entering the target cells, cell nuclei, or membranes at a useful rate. In certain preferred embodiments, the transport molecules of the invention are useful for delivery of proteins or peptides, such as regulatory factors, enzymes, antibodies, drugs or toxins, as well as DNA or RNA, into the cell nucleus or across membranes. Particularly preferred cargo molecules include toxins, non-limiting examples of which include botulinum, waglerin, and tetanus toxins. Intracellular delivery of cargo molecules according to this invention is accomplished by administration of conjugates of the novel transport molecules and cargo molecules to the cells of interest. In other embodiments, the invention provides methods of delivery of cargo molecules across membranes, by administering a transport molecule/cargo molecule conjugate to the membranes of interest. In one particularly preferred embodiment, the transport molecule/cargo molecule conjugate is topically administered to provide for transdermal penetration of the cargo molecule of interest.

### BRIEF DESCRIPTION OF THE DRAWINGS

FIG. 1: In vitro percutaneous penetration of ¹²⁵I-associated radioactivity in human skin with K15RT2, fifteen (15) lysine with a polypeptide corresponding to SEQ ID NO. 1 attached to either end via a G spacer.

### DETAILED DESCRIPTION OF THE INVENTION

### Formation of the Transport Molecules

The preferred transport molecules of this invention are characterized by the presence of a polypeptide having a sequence that corresponds to the reverse sequence of the HIV-TAT basic region amino acid sequence (amino acids 49-57 of naturally-occurring HIV-TAT protein). This reverse sequence of the HIV-TAT basic region, which is RRRQRRKKR (SEQ ID NO. 1) is hereafter referred to as the “reverse-sequence polypeptide”, and may be covalently or non-covalently attached to a cargo molecule of interest to form a conjugate. In certain embodiments, it is advantageous to covalently attach one or more reverse-sequence polypeptides to a cargo molecule of interest, either directly or via a peptide or polymeric linker. For example, the reverse-sequence polypeptide may be advantageously attached to cargo molecules by chemical cross-linking or by genetic fusion, as described herein.

Variants of the reverse-sequence polypeptide are also contemplated by this invention. Generally, any variant of the reverse-sequence polypeptide that can be used in a transport molecule to improve the transdermal or transmembrane penetration of a cargo molecule is considered a part of this invention. For example, in some embodiments, variants of the reverse-sequence polypeptide are produced by the deletion and/or substitution of at least one amino acid present in the reverse-sequence polypeptide to produce a modified reverse-sequence polypeptide. Modified reverse-sequence polypeptide can thus be produced that have amino acid sequences that are substantially similar although not identical, to that of the reverse-sequence polypeptide. Preferred modified reverse-sequence polypeptides include those that are functional equivalent, or functionally equivalent peptide fragments thereof. Such functional equivalents or functionally equivalent fragments possess transmembrane and intracellular penetration ability that is substantially similar to that of naturally-occurring reverse-sequence polypeptide.

Reverse-sequence polypeptides or variants thereof can be obtained using a variety of methods, including genetic engineering techniques or chemical synthesis. In certain preferred embodiments, one or more substitutions may be made to modulate the penetration abilities of the reverse-sequence polypeptide, such that the resulting transport molecule tends to localize in certain areas, such as the cytoplasm of a target cell. Similar behavior has been previously observed for the basic region of naturally occurring HIV-TAT, and has been used to localize or to partially localize the HIV-TAT fragment in the cytoplasm (see e.g., Dang, C. V. and Lee, W. M. F., J. Biol. Chem. 264: 18019-18023 (1989); Hauber, J. et al., J. Virol. 63: 1181-1187 (1989); Ruben, S. A. et al., J. Virol. 63: 1-8 (1989)). Alternatively, a sequence for binding a cytoplasmic component can be attached to the reverse-sequence polypeptide in order to retain the reverse-sequence polypeptide and the cargo molecule in the cytoplasm or to regulate nuclear uptake of a cargo molecule. In other embodiments, cholesterol or other lipid derivatives can be added to the reverse-sequence polypeptide or a variant thereof to increase the membrane solubility of the transport molecule. Of course, delivery of a given cargo molecule to the cytoplasm may be followed by further delivery of the same cargo molecule to the nucleus. Nuclear delivery necessarily involves traversing some portion of the cytoplasm.

While the reverse-sequence polypeptide is useful for providing transmembrane or intracellular delivery of cargo molecules, the transport molecules contemplated by this invention may also contain any other portion of the HIV-TAT native protein that enhances transmembrane or intracellular transport. For example, if desired, the transport molecules may also contain all 86 residues of the full HIV-TAT polypeptide having the sequence of a region of the native HIV-TAT protein that increase sequence of any portion thereof which demonstrates increasing uptake activiting, non-limiting examples of which include residues 1-58, 37-72, or 49-57. However, in preferred embodiments, the transport molecule does not contain the cysteine-rich region of HIV-TAT, which corresponds to amino acids 22-37 of the native HIV-TAT sequence, and in which 7 out of 16 amino acids are cysteine. Those cysteine residues are capable of forming disulfide bonds with each other, with cysteine residues in the cysteine-rich region of other HIV-TAT protein molecules or fragments thereof that may be present, and with cysteine residues that may exist in a protein or polypeptide that constitutes the cargo molecule of interest. Such disulfide bond formation can cause loss of the biological activity of the cargo molecule. Furthermore, even if there is no potential for disulfide bonding to the cargo molecule (for example, when the therapeutic agent is a protein without cysteine residues), disulfide bond formation between transport molecules leads to aggregation and insolubility of the transport molecule, the transport molecule cargo molecule conjugate, or both. Thus, the cysteine-rich region of the native HIV-TAT protein is potentially a source of serious problems in the use of HIV-TAT related proteins for delivery of therapeutic or diagnostic agents. By virtue of the absence of the cysteine-rich region present in HIV-TAT proteins, the preferred transport molecules of this invention avoid the problem of disulfide aggregation, which can result in loss of the biological activity or insolubility of the covalent or non-covalent conjugate of the transport polypeptide/therapeutic agent, or both. Moreover, the reduced size of the preferred transport molecules of this invention also advantageously minimizes interference with the biological activity of the therapeutic or diagnostic agent. A further advantage of the reduced transport molecule size is enhanced uptake efficiency in embodiments of this invention involving attachment of multiple reverse-sequence polypeptides per cargo molecule.

Furthermore, this invention also contemplates transport molecules that contain one or more reverse-sequence polypeptides in conjunction with TAT proteins from other viruses, non-limiting examples of which include HIV-2 (M. Guyader et al., “Genome Organization and Transactivation of the Human Immunodeficiency Virus Type 2”, Nature, 326, pp. 662-669 (1987)), equine infectious anemia virus (R. Carroll et al., “Identification of Lentivirus TAT Functional Domains Through Generation of Equine Infectious Anemia Virus/Human Immunodeficiency Virus Type 1 TAT Gene Chimeras”, J. Virol., 65, pp. 3460-67 (1991)), and simian immunodeficiency virus (L. Chakrabarti et al., “Sequence of Simian Immunodeficiency Virus from Macaque and Its Relationship to Other Human and Simian Retroviruses”, Nature, 328, pp. 543-47 (1987); S. K. Arya et al., “New Human and Simian HIV-Related Retroviruses Possess Functional Transactivator (tat) Gene”, Nature, 328, pp. 548-550 (1987)). It should be understood that transport molecules that comprise the reverse-sequence polypeptide and any polypeptide derived from these other TAT proteins fall within the scope of the present invention, including those characterized by the presence of the TAT basic region and the absence of the TAT cysteine-rich region.

The transport molecules of this invention may be chemically synthesized or produced by recombinant DNA methods when the transport molecules are polypeptides. Methods for chemical synthesis or recombinant DNA production of polypeptides having a known amino acid sequence are well known. Automated equipment for polypeptide or DNA synthesis is commercially available. Host cells, cloning vectors, DNA expression control sequences and oligonucleotide linkers are also commercially available for preparing polypeptide transport molecules.

According to the invention, a cargo molecule is combined, either covalently or non-covalently, with a transport molecule to form a conjugate. In preferred embodiments, the cargo molecules contemplated by the invention include any substance that has prophylactic, therapeutic, or diagnostic application. However, any biologically active agent is also contemplated by this invention, including cargo molecules that can have an adverse affect on the recipient, such as a toxin that is useful for euthanizing animals. Wide latitude exists in the selection of cargo molecules for use in the practice of this invention. Non-limiting examples of cargo molecules contemplated by this invention include drugs, diagnostic agents, enzymes, proteins, polypeptides, oligonucleotides, antigens, and toxins. Cargo molecules contemplated by the invention can be obtained or produced using known techniques, such as chemical synthesis, genetic engineering methods, or isolation from sources in which it occurs naturally.

In one preferred embodiment, the cargo molecule is a toxin molecule derived from a serotype of botulinum toxin. Particularly preferred are toxins directly isolated from botulinum serotypes A, B, C, D, E, F, and G, although modified forms of these botulinum serotypes are also expressly considered to be a part of this invention. Such modified forms include, without limitation, toxin molecules in which contain additions or deletions of amino acid residues, provided that those additions or deletions do not substantially alter the biological effect of the toxin molecule. In other embodiments, the cargo molecule is an antigen and the conjugation to the transport molecule is for the purpose of making a vaccine. For example, the cargo molecule can be an antigen from the bacteria or virus or other infectious agent that the vaccine is to immunize against (e.g. gp120 of HIV). Providing the antigen into the cell cytoplasm allows the cell to process the molecule and express it on the cell surface. Expression of the antigen on the cell surface will raise a killer T-lymphocyte response, thereby inducing immunity.

In yet another embodiment of the invention, the cargo molecule is a protein, such as an enzyme, antibody, toxin, or regulatory factor (e.g., transcription factor) whose delivery into cells, and particularly into the cell nucleus is desired. For example, some viral oncogenes inappropriately turn on expression of cellular genes by binding to their promoters. By providing a competing binding protein in the cell nucleus, viral oncogene-activity can be inhibited.

In a further embodiment, the cargo molecule is a nucleotide sequence to be used as a diagnostic tool (or probe), or as a therapeutic agent, such as an oligonucleotide sequence that is complementary to a target cellular gene or gene region and capable of inhibiting activity of the cellular gene or gene region by hybridizing with it. The rate at which single-stranded and double-stranded nucleic acids enter cells, in vitro and in vivo, may be advantageously enhanced, using the transport molecules of this invention. For example, methods for chemical cross-linking of polypeptides to nucleic acids are well known in the art. In a preferred embodiment of this invention, the cargo molecule is a single-stranded antisense nucleic acid. Antisense nucleic acids are useful for inhibiting cellular expression of sequences to which they are complementary. In another embodiment of this invention, the cargo molecule is a double-stranded nucleic acid comprising a binding site recognized by a nucleic acid-binding protein. An example of such a nucleic acid-binding protein is a viral trans-activator.

The cargo molecule of interest may also be a drug, such as a peptide analog or small molecule enzyme inhibitor, whose introduction specifically and reliably into a cell nucleus is desired.

The cargo molecules of this invention may also be diagnostic agents that provide information, in vitro or in vivo, about the local environment where the cargo molecules are present. Factors to be considered in selecting diagnostic agents include, but are not limited to, the type of experimental information sought, the condition being diagnosed or imaged, the route of administration, non-toxicity, convenience of detection, quantifiability of detection, and availability. Many such diagnostic agents are known to those skilled in the art. Non-limiting examples of suitable diagnostic agents include radiopaque contrast agents, paramagnetic contrast agents, superparamagnetic contrast agents, CT contrast agents and other contrast agents. For example, radiopaque contrast agents (for X-ray imaging) will include inorganic and organic iodine compounds (e.g., diatrizoate), radiopaque metals and their salts (e.g., silver, gold, platinum and the like) and other radiopaque compounds (e.g., calcium salts, barium salts such as barium sulfate, tantalum and tantalum oxide). Suitable paramagnetic contrast agents (for MR imaging) include gadolinium diethylene triaminepentaacetic acid (Gd-DTPA) and its derivatives, and other gadolinium, manganese, iron, dysprosium, copper, europium, erbium, chromium, nickel and cobalt complexes, including complexes with 1,4,7,10-tetraazacyclododecane-N,N′,N″,N′″-tetraacetic acid (DOTA), ethylenediaminetetraacetic acid (EDTA), 1,4,7,10-tetraazacyclododecane-N,-N′,N″-triacetic acid (DO3A), 1,4,7-triazacyclononane-N,N′,N″-triacetic acid (NOTA), 1,4,8,10-tetraazacyclotetradecane-N,N′,N″,N′″-tetraacetic acid (TETA), hydroxybenzylethylene-diamine diacetic acid (HBED) and the like. Suitable superparamagnetic contrast agents (for MR imaging) include magnetites, superparamagnetic iron oxides, monocrystalline iron oxides, particularly complexed forms of each of these agents that can be covalently or noncovalently attached to a reverse-sequence polypeptide or a positively charged backbone that contains a reverse-sequence polypeptide, as described herein. Still other suitable imaging agents are the CT contrast agents including iodinated and noniodinated and ionic and nonionic CT contrast agents, as well as contrast agents such as spin-labels or other diagnostically effective agents.

Other examples of diagnostic agents include marker genes that encode proteins that are readily detectable when expressed in a cell, including, but not limited to, β-galactosidase, green fluorescent protein, blue fluorescent protein, luciferase, and the like. A wide variety of labels may be employed, such as radionuclides, fluors, enzymes, enzyme substrates, enzyme cofactors, enzyme inhibitors, ligands (particularly haptens), and the like. Still other useful substances are those labeled with radioactive species or components, such as ^{99m}Tc glucoheptonate.

The attachment of the cargo molecule to the transport molecule may be effected by any means that produces a link between the two constituents which is sufficiently stable to withstand the conditions used and which does not alter the function of either constituent. The link between them may be non-covalent or covalent. For example, recombinant techniques can be used to covalently attach transporter molecules that are polypeptides to protein/polypeptide-based cargo molecules, by joining the gene coding for the cargo molecule with the gene coding for the polypeptide transporter molecule and then introducing the resulting gene construct into a cell capable of expressing the conjugate. Alternatively, the two separate nucleotide sequences can be expressed in a cell or can be synthesized chemically and subsequently joined covalently, using known techniques. Also, the protein/peptide-based cargo molecule conjugate with the transporter molecule can be synthesized chemically as a single amino acid sequence (i.e., one in which both constituents are present) and, thus, joining is not needed.

Numerous chemical cross-linking methods are known and potentially applicable for conjugating the transport polypeptides of this invention to cargo molecules that are macromolecules. Many known chemical cross-linking methods are non-specific, i.e., they do not direct the point of coupling to any particular site on the transport polypeptide or cargo macromolecule. As a result, use of non-specific cross-linking agents may attack functional sites or sterically block active sites, rendering the conjugated proteins biologically inactive.

A preferred approach to increasing coupling specificity in the practice of this invention is direct chemical coupling to a functional group found only once or a few times in one or both of the polypeptides to be cross-linked. For example, in many proteins, cysteine, which is the only protein amino acid containing a thiol group, occurs only a few times. Also, for example, if a polypeptide contains no lysine residues, a cross-linking reagent specific for primary amines will be selective for the amino terminus of that polypeptide. Successful utilization of this approach to increase coupling specificity requires that the polypeptide have the suitably rare and reactive residues in areas of the molecule that may be altered without loss of the molecule's biological activity.

Cysteine residues may be replaced when they occur in parts of a polypeptide sequence where their participation in a cross-linking reaction would likely interfere with biological activity. When a cysteine residue is replaced, it is typically desirable to minimize resulting changes in polypeptide folding. Changes in polypeptide folding are minimized when the replacement is chemically and sterically similar to cysteine. For these reasons, serine is preferred as a replacement for cysteine. A cysteine residue may be introduced into a polypeptide's amino acid sequence for cross-linking purposes. When a cysteine residue is introduced, introduction at or near the amino or carboxy terminus is preferred. Conventional methods are available for such amino acid sequence modifications, whether the polypeptide of interest is produced by chemical synthesis or expression of recombinant DNA.

Coupling of the two constituents can be accomplished via a coupling or conjugating agent. There are several intermolecular cross-linking reagents that can be utilized (see, for example, Means, G. E. and Feeney, R. E., Chemical Modification of Proteins, Holden-Day, 1974, pp. 39-43). Among these reagents are, for example, J-succinimidyl 3-(2-pyridyldithio) propionate (SPDP) or N,N′-(1,3-phenylene) bismaleimide (both of which are highly specific for sulfhydryl groups and form irreversible linkages); N,N′-ethylene-bis-(iodoacetamide) or other such reagent having 6 to 11 carbon methylene bridges (which relatively specific for sulfhydryl groups); and 1,5-difluoro-2,4-dinitrobenzene (which forms irreversible linkages with amino and tyrosine groups). Other cross-linking reagents useful for this purpose include: p,p′-difluoro-m,m′dinitrodiphenylsulfone (which forms irreversible cross-linkages with amino and phenolic groups); dimethyl adipimidate (which is specific for amino groups); phenol-1,4-disulfonylchloride (which reacts principally with amino groups); hexamethylenediisocyanate or diisothiocyanate, or azophenyl-p-diisocyanate (which reacts principally with amino groups); glutaraldehyde (which reacts with several different side chains) and disdiazobenzidine (which reacts primarily with tyrosine and histidine).

Cross-linking reagents may be homobifunctional, i.e., having two functional groups that undergo the same reaction. A preferred homobifunctional cross-linking reagent is bismaleimidohexane (“BMH”). BMH contains two maleimide functional groups, which react specifically with sulfhydryl-containing compounds under mild conditions (pH 6.5-7.7). The two maleimide groups are connected by a hydrocarbon chain. Therefore, BMH is useful for irreversible cross-linking of polypeptides that contain cysteine residues.

Cross-linking reagents may also be heterobifunctional. Heterobifunctional cross-linking agents have two different functional groups, for example an amine-reactive group and a thiol-reactive group, that will cross-link two proteins having free amines and thiols, respectively. Examples of heterobifunctional cross-linking agents are succinimidyl 4-(N-maleimidomethyl)cyclohexane-1-carboxylate (“SSMCC”), m-maleimidobenzoyl-N-hydroxysuccinimide ester (“MBS”), and succinimide 4-(p-maleimidophenyl)butyrate (“SMPB”), an extended chain analog of MBS. The succinimidyl group of these cross-linkers reacts with a primary amine, and the thiol-reactive maleimide forms a covalent bond with the thiol of a cysteine residue.

Cross-linking reagents often have low solubility in water. A hydrophilic moiety, such as a sulfonate group, may be added to the cross-linking reagent to improve its water solubility. Sulfo-MBS and sulfo-SMCC are examples of cross-linking reagents modified for water solubility.

Many cross-linking reagents yield a conjugate that is essentially non-cleavable under cellular conditions. However, some cross-linking reagents contain a covalent bond, such as a disulfide, that is cleavable under cellular conditions. For example, dithiobis(succinimidylpropionate) (“DSP”), Traut's reagent and N-succinimidyl 3-(2-pyridyldithio) propionate (“SPDP”) are well-known cleavable cross-linkers. The use of a cleavable cross-linking reagent permits the cargo moiety to separate from the transport polypeptide after delivery into the target cell. Direct disulfide linkage may also be useful.

Some new cross-linking reagents such as n-γ-maleimidobutyryloxy-succinimide ester (“GMBS”) and sulfo-GMBS, have reduced immunogenicity. In some embodiments of the present invention, such reduced immunogenicity may be advantageous.

Numerous cross-linking reagents, including the ones discussed above, are commercially available. Detailed instructions for their use are readily available from the commercial suppliers. A general reference on protein cross-linking and conjugate preparation is: S. S. Wong, Chemistry of Protein Conjugation and Cross-Linking, CRC Press (1991).

Chemical cross-linking may include the use of spacer arms. Spacer arms provide intramolecular flexibility or adjust intramolecular distances between conjugated moieties and thereby may help preserve biological activity. A spacer arm may be in the form of a polypeptide moiety comprising spacer amino acids. In one particular embodiment, the spacer linker is composed of one or more glycine units, such as a GG dimer, for example. Alternatively, a spacer arm may be part of the cross-linking reagent, such as in “long-chain SPDP” (Pierce Chem. Co., Rockford, Ill., cat. No. 21651H).

It will be recognized by those of ordinary skill in the art that when the transport polypeptide is genetically fused to the cargo moiety, it is advantageous to add an amino-terminal methionine, but spacer amino acids (e.g., CysGlyGly or GlyGlyCys) need not be added in some embodiments. A unique terminal cysteine residue is a preferred means of chemical cross-linking. According to some preferred embodiments of this invention, the carboxy terminus of the reverse-sequence polypeptide is genetically fused to the amino terminus of a cargo molecule that includes a polypeptide or protein.

In certain preferred embodiments, the reverse-sequence polypeptide is itself a transport molecule that non-covalently associates with a cargo molecule to form a non-covalent conjugate that enhances delivery of the cargo molecule. Alternatively, the reverse-sequence polypeptide is covalently attached, not to the cargo molecule, but instead to a backbone molecule (either directly or via a linker) to form a transport molecule that non-covalently associates with the cargo molecule to form a conjugate. In a particularly preferred embodiment, the transport molecule includes one or more copies of the reverse-sequence polypeptide, covalently attached to a positively-charged backbone. Optionally, other transport-enhancing fragments of native HIV-TAT or of TAT proteins from other viruses may be attached to the positively charged backbone as well. A positively-charged backbone is typically a linear chain of atoms, either with groups in the chain carrying a positive charge at physiological pH, or with groups carrying a positive charge attached to side chains extending from the backbone. The linear backbone is a hydrocarbon backbone, which is, in some embodiments, interrupted by heteroatoms selected from nitrogen, oxygen, sulfur, silicon and phosphorus. The majority of backbone chain atoms are usually carbon. Additionally, the backbone will often be a polymer of repeating units (e.g., amino acids, poly(ethyleneoxy), poly(propyleneamine), and the like). In one group of embodiments, the positively charged backbone is a polypropyleneamine wherein a number of the amine nitrogen atoms are present as ammonium groups (tetra-substituted) carrying a positive charge. In another group of embodiments, the backbone has attached a plurality of sidechain moieties that include positively charged groups (e.g., ammonium groups, pyridinium groups, phosphonium groups, sulfonium groups, guanidinium groups, or amidinium groups). The sidechain moieties in this group of embodiments can be placed at spacings along the backbone that are consistent in separations or variable. Additionally, the length of the sidechains can be similar or dissimilar. For example, in one group of embodiments, the sidechains can be linear or branched hydrocarbon chains having from one to twenty carbon atoms and terminating at the distal end (away from the backbone) in one of the above-noted positively charged groups.

In one group of embodiments, the positively charged backbone is a polypeptide having multiple positively charged sidechain groups (e.g., lysine, arginine, ornithine, homoarginine, and the like). One of skill in the art will appreciate that when amino acids are used in this portion of the invention, the sidechains can have either the D- or L-form (R or S configuration) at the center of attachment.

Alternatively, the backbone can be an analog of a polypeptide such as a peptoid. See, for example, Kessler, Angew. Chem. Int. Ed. Engl. 32:543 (1993); Zuckermann et al. Chemtracts-Macromol. Chem. 4:80 (1992); and Simon et al. Proc. Nat'l. Acad. Sci. USA 89:9367 (1992)). Briefly, a peptoid is a polyglycine in which the sidechain is attached to the backbone nitrogen atoms rather than the α-carbon atoms. As above, a portion of the sidechains will typically terminate in a positively charged group to provide a positively charged backbone component. Synthesis of peptoids is described in, for example, U.S. Pat. No. 5,877,278. As the term is used herein, positively charged backbones that have a peptoid backbone construction are considered “non-peptide” as they are not composed of amino acids having naturally occurring sidechains at the .alpha.-carbon locations.

A variety of other backbones can be used employing, for example, steric or electronic mimics of polypeptides wherein the amide linkages of the peptide are replaced with surrogates such as ester linkages, thioamides (—CSNH—), reversed thioamide (—NHCS—), aminomethylene (—NHCH₂—) or the reversed methyleneamino (—CH₂NH—) groups, keto-methylene (—COCH₂—) groups, phosphinate (—PO₂RCH₂—), phosphonamidate and phosphonamidate ester (—PO₂RNH—), reverse peptide (—NHCO—), trans-alkene (—CR═CH—), fluoroalkene (—CF═CH—), dimethylene (—CH₂CH₂—), thioether (—CH₂S—), hydroxyethylene (—CH(OH)CH₂—), methyleneoxy (—CH₂O—), tetrazole (CN₄), sulfonamido (—SO₂NH—), methylenesulfonamido (—CHRSO₂NH—), reversed sulfonamide (—NHSO₂—), and backbones with malonate and/or gem-diamino-alkyl subunits, for example, as reviewed by Fletcher et al. ((1998) Chem. Rev. 98:763) and detailed by references cited therein. Many of the foregoing substitutions result in approximately isosteric polymer backbones relative to backbones formed from α-amino acids.

In another particularly preferred embodiment, the backbone portion is a polylysine and the reverse-sequence polypeptides are attached to the lysine sidechain amino groups. The polylysine used in this particularly preferred embodiment can be any of the commercially available (Sigma Chemical Company, St. Louis, Mo., USA) polylysines such as, for example, polylysine having MW>70,000, polylysine having MW of 70,000 to 150,000, polylysine having MW 150,000 to 300,000 and polylysine having MW>300,000. The appropriate selection of a polylysine will depend on the remaining components of the composition and will be sufficient to provide an overall net positive charge to the composition.

### Delivery of the Transport Moleculel/Cargo Molecule Conjugate

This invention is generally applicable for therapeutic, prophylactic or diagnostic intracellular or transmembrane delivery of small molecules and macromolecules, such as proteins, nucleic acids and polysaccharides, that are not inherently capable of entering target cells or penetrating biological membranes at a useful rate. The processes and compositions of this invention may be applied to any organism, including humans. The processes and compositions of this invention may also be applied to animals and humans in utero. According to one preferred embodiment of this invention, a cargo molecule is delivered into the cells of various organs and tissues following introduction of a transport molecule-cargo conjugate into or onto a live human or animal. For example, the cargo molecule/transport molecule conjugate may be brought into contact with cells into which introduction of the cargo molecule is desired. As a result, the conjugate enters into cells, passing into the nucleus. In another embodiment, the cargo molecule/transport molecule conjugate is administered to a surface of a membrane to cause transmembrane penetration of the cargo molecule/transport molecule conjugate. For example, the cargo molecule/transport molecule conjugate may be administered topically to a region that would benefit from the therapeutic action of the cargo molecule. In a particularly preferred embodiment, the cargo molecule is a serotype of botulinum toxin, and the cargo molecule/transport molecule conjugate is topically administered in regions of the skin having furrows or wrinkles, in order to reduce the appearance of the furrows or wrinkles.

Alternatively, the cargo molecule/transporter molecule conjugate can be delivered in vivo by cells that are produced and implanted into an individual. The cells are genetically engineered so that they express the cargo molecule/transport molecule conjugate continuously in vivo.

Alternatively, the present invention may be used to deliver a cargo molecule in vitro. For example, in in vitro applications in which the cargo molecule is to be delivered into cells in culture, the cargo molecule/transport molecule conjugate can be simply added to the culture medium. This is useful, for example, as a means of delivering into the nucleus substances whose effect on cell function is to be assessed. For example, the activity of purified transcription factors can be measured, or the in vitro assay can be used to provide an important test of a cargo molecule's activity, prior to its use in in vivo treatment.

Delivery can also be carried out in vitro by producing cells that synthesize the desired cargo molecule/transport molecule conjugate in vitro or by combining a sample (e.g., blood, bone marrow) obtained from an individual with the cargo molecule/transport conjugate, under appropriate conditions. For example, a selected cargo molecule in combination with TAT protein or the cargo molecule of interest-TAT protein conjugate can be combined with a sample obtained from an individual (e.g., blood, bone marrow) in order to introduce the molecule of interest into cells present in the sample and, after treatment in this manner, the sample returned to the individual. A series of treatments carried out in this manner can be used to prevent or inhibit the effects of an infectious agent. For example, blood can be removed from an individual infected with HIV or other viruses, or from an individual with a genetic defect. The blood can then be combined with the cargo molecule/transport molecule conjugate in which the cargo molecule of interest is a drug capable of inactivating the virus, or an oligonucleotide sequence capable of hybridizing to a selected virus sequence and inactivating, it or a protein that supplements a missing or defective protein, under conditions appropriate for entry in cells of the conjugate and maintenance of the sample in such a condition that it can be returned to the individual. After treatment, the blood is returned to the individual.

Delivery can be carried out in vivo by administering the cargo molecule/transport molecule conjugate to an individual in whom it is to be used for diagnostic, preventative or therapeutic purposes. The target cells may be in vivo cells, i.e., cells composing the organs or tissues of living animals or humans, or microorganisms found in living animals or humans.

In some embodiments, the transport molecule/cargo molecule conjugate is combined with an agent that increases stability and penetration. For example, metal ions that bind to HIV-TAT protein and increase its stability and penetration, can be used for this purpose. Alternatively, a lysosomotrophic agent is provided extracellularly in conjunction with the transport molecule and cargo molecule in order to enhance uptake by cells. The lysosomotrophic agent can be used alone or in conjunction with a stabilizer. For example, lysosomotrophic agents such as chloroquine, monensin, amantadine and methylamine, which have been shown to increase uptake of naturally-occurring HIV-TAT in some cells by a few hundred fold, can be used for this purpose.

In another embodiment, a basic peptide, such as a peptide sequence that corresponds to residues 38-58 or HIV-TAT or protamine, is provided extracellularly with the transport molecule and cargo molecule to enhance the uptake of the cargo moleccule. Such basic peptides can also be used alone, or in combination with stabilizing agents or lysosomotrophic agents.

The pharmaceutical compositions of this invention may be for therapeutic, prophylactic or diagnostic applications, and may be in a variety of forms. These include, for example, solid, semi-solid, and liquid dosage forms, such as tablets, pills, powders, liquid solutions or suspensions, aerosols, liposomes, suppositories, injectable and infusible solutions and sustained release forms. The preferred form depends on the intended mode of administration and the therapeutic, prophylactic or diagnostic application. According to this invention, a selected cargo molecule/transport molecule conjugate may be administered by conventional routes of administration, such as parenteral, subcutaneous, intravenous, intramuscular, intralesional, intrasternal, intracranial or aerosol routes. Topical routes of administration may also be used, with application of the compositions locally to a particular part of the body (e.g., skin, lower intestinal tract, vagina, rectum) where appropriate. The compositions also preferably include conventional pharmaceutically acceptable carriers and adjuvants that are known to those of skill in the art.

Generally, the pharmaceutical compositions of the present invention may be formulated and administered using methods and compositions similar to those used for pharmaceutically important polypeptides such as, for example, alpha interferon. It will be understood that conventional doses will vary depending upon the particular cargo molecule involved, as well as the patient's health, weight, age, sex, the condition or disease and the desired mode of administration. The pharmaceutical compositions of this invention include pharmacologically appropriate carriers, adjuvants and vehicles. In general, these carriers include aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. In addition, intravenous vehicles can include fluid and nutrient replenishers, and electrolyte replenishers, such as those based on Ringer's dextrose. Preservatives and other additives can also be present, such as, for example, antimicrobials, antioxidants, chelating agents, and inert gases. See, generally, Remington's Pharmaceutical Sciences, 16th Ed., Mack, ed. 1980.

It should be appreciated, however, that alternate embodiments of this invention are not limited to clinical applications. This invention may be advantageously applied in medical and biological research. In research applications of this invention, the cargo molecule may be a drug or a diagnostic agent. Transport molecules of this invention may be used as research laboratory reagents, either alone or as part of a transport molecule conjugation kit.

While we have described a number of embodiments of this invention, it is apparent that our basic constructions can be altered to provide other embodiments that utilize the processes and products of this invention. Therefore, it will be appreciated that the scope of this invention is to be defined by the appended claims rather than by the specific embodiments that have been presented by way of example.

### EXAMPLE 1

The objective of the present study was to evaluate the possibility of delivering a large cargo molecule (botulinum toxin type A) to human skin in vitro using flow-through diffusion cells. Since the toxin is of considerable size, the dermal uptake without use of any transport molecule was expected to be negligibly low. Therefore, a carrier solution was added at different toxin/carrier ratios in an attempt to increase/facilitate dermal uptake through the stratum corneum layer. In addition to the amount present in the receptor fluid at various time points, the distribution in the various skin layers was evaluated after 24 hours. The complete Neuronox® product (i.e. the toxin/albumin complex including accessory proteins) were radio-labelled using ¹²⁵I.

### 1.1 Test System

Preparation of Skin Membranes: Human Skin Membranes were Prepared from frozen skin sample (a single donor directly after abdominal surgery). After thawing, the skin was dermatomed using a Dermatome 25 mm (Nouvag GmbH, Germany) to a recorded thickness of approximately 400 μm.

Flow-through diffusion cells: The skin membranes were placed in 9 mm flow-through automated diffusion cells (PermeGear Inc., Riegelsville, Pa., USA). The skin surface temperature was kept at approximately 32° C., at ambient humidity. The receptor fluid was pumped at a speed of about 1.6 mL h-1 and consisted of Phosphate Buffered Saline (PBS) containing 0.01% sodium azide (w/v).

### 1.2 Experimental Design and Procedures

¹²⁵I-labelling of the test substances: The contents of one vial containing the Neuronox product was reconstituted in 100 μL 50 mM KH₂PO₄ buffer, pH 7.2. During iodination 37 MBq Na¹²⁵I (10 μl), 20 μl of an approximately 100,000-fold diluted hydrogen peroxide solution in water (30% (v/v) perhydrol) and 20 μl lactoperoxidase. (4 μg.10 μL-1 water) was added to the vial containing the Neuronox® product. After about 60 seconds, iodination was stopped by the addition of 50 μL tyrosine solution (1 mg mL-1) in phosphate buffer to remove excess ¹²⁵I that had not yet reacted with the available proteins (toxin, albumin etc.). After one minute, 125I (bound to L-tyrosine) was separated from the radio-labelled proteins by using a Sephadex G25 fine column of about 10 mL volume equilibrated with assay buffer containing 0.5% (w/v) BSA. Fractions of about 250 μL were collected. Sub-fractions were taken for radio-activity measurement. Fractions were stored at 2-10° C. until further use.

Experimental design: The Neuronox® product was evaluated for its ability to penetrate the skin in K15RT2 carrier solutions. Prior to application of the test compound, the skin integrity was assessed by determining the permeability coefficient (Kp) of tritiated water. The experimental set up was as follows:

| Group | n | Carrier/toxin ratio | Test substance |
|---|---|---|---|
| A | 4 | Control (no carrier) | NNX alone |
| B | 5 | 1.1:1 | K15TR2 + NNX |

Recovery procedure: After 24 hours, the unabsorbed test substance (dislodgeable dose) was removed from the application site using a mild soap solution (3% Teepol in water) and cotton swabs. The skin surface was dried after washing using dry cotton swabs. The receptor compartment and the donor compartment were rinsed with water (2 times 1 mL). Subsequently, each skin membrane was tape stripped (10 times per membrane) using D-squame (Monaderm, Monaco). Tape stripping was discontinued in case the epidermis was ruptured. Tape strips containing (pieces of) the epidermis were pooled with the skin membrane (epidermis). Finally, the epidermis and dermis were separated mechanically using a scalpel knife and tweezers. Radioactivity in all fractions was measured by γ-radiation counting.

### 1.3 Analysis

Determination of radioactivity: Radioactivity in the samples of the integrity test was determined by liquid scintillation counting (LSC) using DOT-DPMTM (digital overlay technique using the spectrum library and the external standard spectrum) for quench correction on a Wallac Pharmacia model S1414 scintillation counter. Calibration procedures for the instruments are established at the testing facilities.

Dose formulations: Aliquots of the dose formulation taken just before and directly after dosing were added directly to liquid scintillant (Ultima Gold™) and measured by LSC. Receptor fluid Samples of the receptor fluid were added directly to a liquid scintillant (Ultima Gold™) and measured by LSC. Radioactivity in the samples of the absorption test using ¹²⁵I-labelled test compound were determined using a Gamma Counter (Perkin Elmer).

### 1.4 Calculations

The total absorption is defined as the amount of compound-related radioactivity present in the receptor fluid, the receptor compartment wash, and the skin (excluding tape strips)

### 2. Results

Percutaneous Absorption of the Test Item

The percutaneous absorption of [¹²⁵I]Neuronox®, was evaluated on human skin membranes. The exposure time was 24 hours. The (tissue) distribution is presented in table 1.

TABLE 1
Overview table of the in vitro percutaneous penetration of
¹²⁵I-associated radioactiviy in human skin (expressed as
percentage of the applied dose)

| | |
|---|---|
| A | B |

| Group | mean | sd | mean | sd |
|---|---|---|---|---|
| Skin wash | 111.15 | 2.10 | 101.44 | 3.51 |
| Charcoal filter | 0.01 | 0.00 | 0.01 | 0.00 |
| Stratum Cornea | 0.76 | 0.59 | 2.43 | 1.43 |
| Epidermis | 0.28 | 0.09 | 0.30 | 0.09 |
| Dermis | 0.27 | 0.41 | 1.03 | 0.77 |
| Receptor fluid | 0.43 | 0.38 | 0.93 | 0.72 |
| Total recovery | 112.94 | 1.14 | 106.28 | 1.52 |

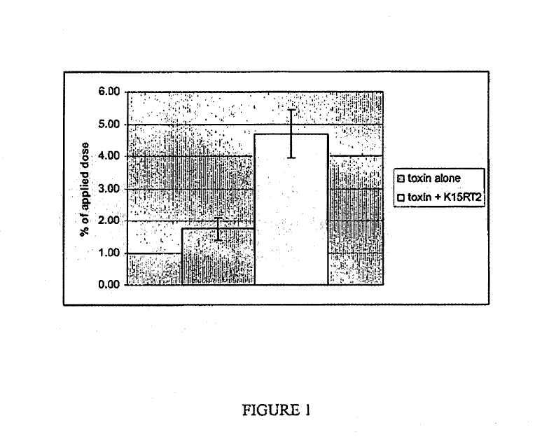

FIG. 1 shows the in vitro percutaneous penetration of ¹²⁵I-associated radioactivity in human skin with K15RT2. The data clearly show that the in vitro percutaneous penetration of ¹²⁵I-associated radioactivity is much higher with botulinum toxin plus K15RT2, as compared to botulinum toxin alone.

We claim:

1. A transport molecule for the delivery of a cargo molecule, wherein said transport molecule comprises a reverse-sequence polypeptide having an amino acid sequence according to SEQ ID NO 1.

2. The transport molecule according to claim 1, wherein the reverse-sequence polypeptide is covalently attached to the cargo molecule.

3. The transport molecule according to claim 1, wherein the reverse-sequence polypeptide is covalently bound to a positively-charged backbone that is non-covalently attached to the cargo molecule.

4. The transport molecule according to claim 3, wherein the reverse-sequence polypeptide is covalently bound to a positively charged backbone that is non-covalently bound to the cargo molecule.

5. The transport molecule according to claim 1, wherein said transport molecule increases the penetration of the cargo molecule through a biological membrane.

6. The transport molecule according to claim 5, wherein the biological membrane is found in the skin.

7. The transport molecule of claim 1, wherein said transport molecule increases the intracellular penetration of the cargo molecule.

8. A conjugate for the delivery of a cargo molecule, said conjugate comprising
a transport molecule comprising a reverse-sequence polypeptide having an amino acid sequence as set forth in SEQ ID NO. 1;
and a cargo molecule.

9. The conjugate according to claim 8, wherein said transport molecule is covalently attached to said cargo molecule.

10. The conjugate according to claim 8, wherein said transport molecule is non-covalently attached to said cargo molecule.

11. The conjugate according to claim 8, wherein said cargo molecule is a therapeutic agent.

12. The conjugate according to claim 11, wherein said therapeutic agent is selected from the group consisting of peptides, proteins, oligonucleotides, enzymes, and antigens.

13. The conjugate according to claim 11, wherein said therapeutic agent derived from a serotype of botulinum toxin or a fragment thereof.

14. The conjugate according to claim 13, wherein said diagnostic agent is selected from the group consisting of radiopaque contrast agents, paramagnetic contrast agents, superparamagnetic contrast agents, and CT contrast agents.

15. A method for the treatment of a disease, wherein said method comprises
selecting a cargo molecule;
selecting a transport molecule;
binding said cargo molecule to said transport molecule either covalently or non-covalently to form a cargo molecule/transport molecule conjugate; and
administering said conjugate to target cells or to a membrane in order to cause intracellular or transmembrane delivery of the cargo molecule.


SEQUENCE LISTING (1 sequence)

SEQ ID NO: 1 - protein, 9 aa, Human immunodeficiency virus
RRRQRRKKR